(19) United States
(12) Patent Application Publication
(10) Pub. No.: US 2023/0265532 A1
(43) Pub. Date: Aug. 24, 2023

(54) RAPID AND HIGHLY SENSITIVE LUMINESCENT BIOMOLECULE DETECTION

(71) Applicant: Ascella Biosystems, Inc., San Francisco, CA (US)

(72) Inventors: Lucian ORBAI, San Francisco, CA (US); Deepak BOGGAVARAPU, San Francisco, CA (US)

(21) Appl. No.: 18/193,590

(22) Filed: Mar. 30, 2023

Related U.S. Application Data
(63) Continuation of application No. PCT/US2021/053022, filed on Sep. 30, 2021.
(60) Provisional application No. 63/085,621, filed on Sep. 30, 2020.

(51) Int. Cl. C12Q 1/70; C12Q 1/682; B01L 3/00

(52) U.S. Cl. CPC C12Q 1/701; B01L 3/502715; C12Q 1/682; B01L 2200/04; B01L 2200/16; B01L 2200/026; B01L 2300/042; B01L 2300/044; B01L 2300/0654

(57) ABSTRACT

Provided are methods, compositions and devices for high sensitivity detection of biomolecules such as nucleic acids in biological samples. The methods rely on target detection, nucleic acid amplification, and sensitive detection to provide a signal which can be conveniently measured in a lab assay or device, including with portable and point-of-care instrumentation.

### CROSS-REFERENCE

This application is a continuation of International Application No. PCT/US21/53022 filed on Sep. 30, 2021, which claims the benefit of U.S. Provisional Pat. Application No. 63/085,621, filed on Sep. 30, 2020, each of which is entirely incorporated herein by reference.

### SEQUENCE LISTING

The instant application contains a Sequence Listing which has been submitted electronically in XML format and is hereby incorporated by reference in its entirety. Said XML copy, created on Mar. 30, 2023, is named 60496-701.301SL.xml and is 4,968 bytes in size.

### BACKGROUND OF THE INVENTION

Detection of biomolecules, often present at low levels, is of great importance in biology and medicine. For example, diagnosis and monitoring of individuals carrying or suspected of carrying a pathogenic organisms relies on detection of pathogen DNA or RNA.

Currently, state of the art active infection diagnostic methods rely on qPCR/qRT-PCR, which relies on exponential amplification of the generated DNA using PCR and concomitant optical (typically fluorescent) detection. In the case of qRT-PCR, reverse transcription is also required prior to amplification. Quantitative PCR requires complex lab instrumentation and the complexity of the steps involved (including thermal cycling, detection, and result interpretation) means laboratory equipment can take up to 4 hours per run. Further, the exponential nature of the amplification step results in extremely sensitive detection, but also means that qRT-PCR can be prone to artifacts. The Ct values generated cannot be directly interpreted by a user but require expert analysis. Newer isothermal approaches are still too slow (generally 30 mins to 1 h per run) and have reduced sensitivity relative to PCR.

Therefore, there remains a need for highly sensitive and rapid detection of biomolecules such as nucleic acids in complex biological samples.

### SUMMARY OF THE INVENTION

The present invention addresses this need and provides additional advantages.

In one aspect, the invention provides a method of detecting a target nucleic acid sequence, comprising contacting a sample suspected to contain the target nucleic acid sequence with a reaction mixture comprising: i) a first nucleic acid probe comprising a first sequence complementary to a template nucleic acid sequence, and further comprising a sequence P at the 3′ end of the first nucleic acid probe, wherein P is complementary to a sequence Pc within the first nucleic acid probe, and P is annealed to Pc in the absence of target nucleic acid; ii) a nucleic acid template comprising Pc, such that P anneals to Pc upon said contacting; iii) a polymerase capable of extending the 3′ end of the nucleic acid probe; and iv) a nucleotide capable of being incorporated by the polymerase, thereby extending the 3′ end of the first nucleic acid probe; and detecting the activity of the polymerase.

In some embodiments, at least one of the nucleotides is an ATP-linked nucleotide, such that incorporation of the nucleotide by the polymerase results in release of a molecule of ATP. For example, the ATP-linked nucleotide has the formula:

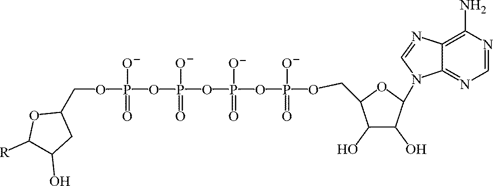

wherein R is a purine, a pyrimidine, or a non-natural base analog. In some embodiments, R is adenine, guanidine, cytidine or thymidine.

In some embodiments, the detecting comprises measuring the amount of ATP generated by the incorporation of the nucleotide by the polymerase. For instance, the ATP is measured by luminescence. In some embodiments, the detecting comprises measuring the amount of pyrophosphate generated by the polymerase. For example, the reagent mixture comprises ATP sulfurylase and/or adenosine 5′-phosphosulfate. In some embodiments, ATP sulfurylase converts phosphosulfate and PPi into ATP, which is then measured by luminescence. In some embodiments, the detection of pyrophosphate is performed electrochemically.

Generally, detecting the activity of the polymerase is performed by measuring a signal proportional to the activity of the polymerase. In some embodiments, the detecting comprises measuring a luminescent signal. For example, the reagent mixture comprises luciferase and a luciferase substrate. In some embodiments, the detecting comprises measuring a fluorescent signal. For instance, the fluorescent signal results from the presence of a nucleic acid binding dye.

The nucleic acid template may be DNA, RNA, or a hybrid. The nucleic acid template may be linear or circular. In some embodiments, the nucleic acid template is a circular oligonucleotide. In some embodiments, the nucleic acid template comprises between 15 and 10000 nt, for example between 15 and 6500 nt; between 15 and 2000 nt; between 15 and 1000 nt; between 15 and 400 nt; between 15 and 200 nt; between 15 and 150 nt; between 15 and 100 nt; or between 15 and 75 nt. In some embodiments, the nucleic acid template is a circular oligonucleotide and comprises between 15 and 10000 nt, for example between 15 and 6500 nt; between 15 and 2000 nt; between 15 and 1000 nt; between 15 and 400 nt; between 15 and 200 nt; between 15 and 150 nt; between 15 and 100 nt; or between 15 and 75 nt.

In some embodiments, the nucleic acid template comprises less than 25, 20, 15, 10, or 5% T bases. In some embodiments, the nucleic acid template comprises no T bases. In some embodiments, the nucleic acid template comprises less than 5% T bases and less than 65% G/C bases. In some embodiments, the nucleic acid template is a circular oligonucleotide and comprises less than 25, 20, 15, 10, or 5% T bases. In some embodiments, the circular oligonucleotide comprises no T bases. In some embodiments, the circular oligonucleotide comprises less than 5% T bases and less than 65% G/C bases.

In some embodiments, the first nucleic acid probe forms a hairpin.

In some embodiments, the contacting step of any method of the invention is performed at room temperature. Alternatively, the contacting is performed at a temperature greater than 37° C. For instance, the temperature is between 42 and 70° C., or between 50 and 65° C.

In some embodiments, the polymerase is a thermostable polymerase.

In some embodiments, the reaction mixture comprises a second nucleic acid probe, wherein the second nucleic acid probe binds to a sequence complementary to that of the circular nucleic acid. In some embodiments, the second nucleic acid probe comprises a sequence P at the 3′ end of the second nucleic acid probe, wherein P is complementary to a sequence Pc within the second nucleic acid probe, and P is annealed to Pc in the absence of first nucleic acid probe which has been extended by polymerase.

In some embodiments, the reaction mixture comprises a hyperbranching primer.

In some embodiments, the reaction mixture further comprises a single-stranded binding protein, for example T4 gene 32 protein.

In some embodiments, prior to the contacting step, the sample is incubated with a reagent that reduces the concentration of ATP. For example, the reagent is apyrase. The reagent, such as apyrase, may be immobilized on a solid support.

In some embodiments, prior to the contacting step, the sample is incubated with a reagent that reduces the concentration of pyrophosphate. For example, the reagent is pyrophosphatase. The reagent, such as pyrophosphatase, may be immobilized on a solid support.

In some embodiments, prior to the contacting step, the sample is incubated with a reagent that lyses a viral particle. In some embodiments, the reagent is a detergent. In some embodiments, the reagent is a non-ionic detergent.

In some embodiments, the target nucleic acid is RNA, for example SARS-CoV-2 RNA.

In a related aspect, the invention also provides devices configured for performing the methods of the invention.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows a nucleic acid probe of the invention binding to a template molecule and initiating a rolling circle reaction at the 3′ end of the probe.

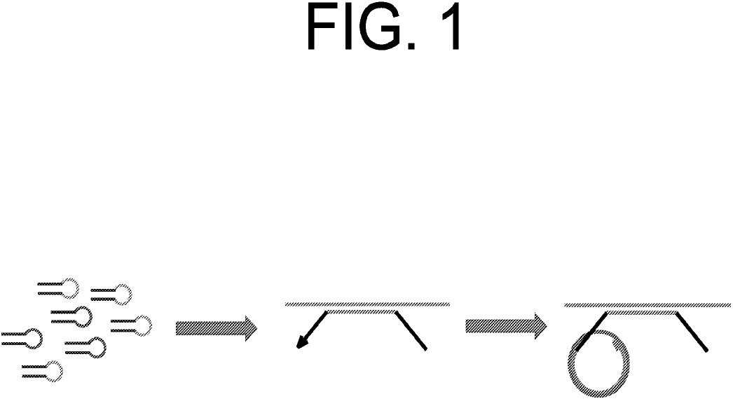

FIG. 2 shows exponential amplification of repeats encoded by the circular oligonucleotide templates.

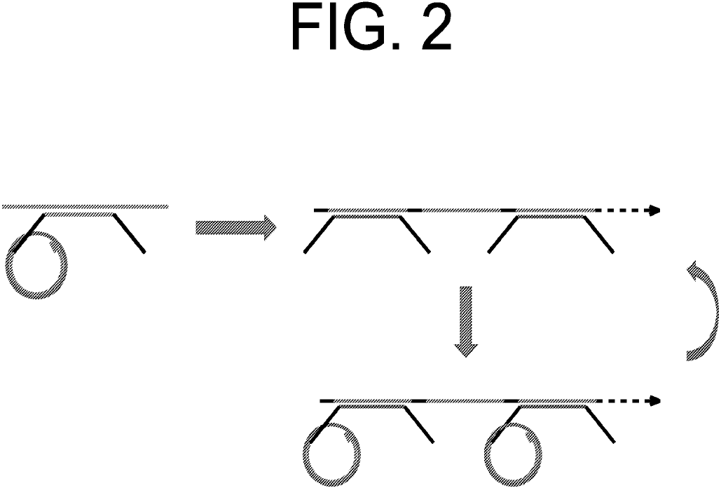

FIG. 3 shows rolling circle amplification of circular oligonucleotide templates using ARN deoxynucleotides and detection using a luciferase/luciferin system.

FIG. 4 shows a cartridge for use with a device of the invention.

FIG. 5 describes the components of a cartridge for use with a device of the invention.

FIG. 6 illustrates a device of the invention with and the process of inserting a cartridge.

### DETAILED DESCRIPTION OF THE INVENTION

The present invention discloses novel methods, compositions, and devices for detection of target biomolecules including in biological samples.

A “target” is a biomolecule or analyte whose presence or concentration in a sample is to be determined, including proteins, antigens, and nucleic acids. Targets can be naturally occurring, i.e. or synthetic. In one aspect, the target is a nucleic acid. Target nucleic acids can be single-stranded or double-stranded, and may be DNA, RNA, or a combination thereof. Target nucleic acids may be purified or isolated, or may be present in a mixture non-purified or non-isolated. Targets of any origin are encompassed. In one aspect, the target nucleic acid is of bacterial or viral origin, whether pathogenic or non-pathogenic. For example, the target nucleic acid is viral DNA, viral RNA, bacterial genomic DNA, bacterial RNA, or bacterial mtDNA. In another aspect, the target nucleic acid is of genomic origin, for example mammalian genomic DNA or transcribed RNA.

As used herein, two nucleic acids or nucleic acid regions “correspond” to one another if they are both complementary to the same nucleic acid sequence. Two nucleic acids or nucleic acid regions are “complementary” to one another if they base-pair with each other to form a double-stranded nucleic acid molecule.

“Hybridization” or “hybridize” or “anneal” refers to the ability of completely or partially complementary nucleic acid strands to come together under specified hybridization conditions in a parallel or preferably antiparallel orientation to form a stable double-stranded structure or region (sometimes called a “hybrid”) in which the two constituent strands are joined by hydrogen bonds. Although hydrogen bonds typically form between adenine and thymine or uracil (A and T or U) or cytosine and guanine (C and G), other base pairs may form (e.g., Adams et al., The Biochemistry of the Nucleic Acids, 11th ed., 1992).

“Substantially homologous” or “substantially corresponding” means a probe, nucleic acid, or oligonucleotide has a sequence of at least 10, 20, 30, 40, 50, 100, 150, 200, 300, 400, or 500 contiguous bases that is at least 80% (preferably at least 85%, 90%, 95%, 96%, 97%, 98%, and 99%, and most preferably 100%) identical to contiguous bases of the same length in a reference sequence. Homology between sequences may be expressed as the number of base mismatches in each set of at least 10 contiguous bases being compared.

“Substantially complementary” means that an oligonucleotide has a sequence containing at least 10, 20, 30, 40, 50, 100, 150, 200, 300, 400, or 500 contiguous bases that are at least 80% (preferably at least 85%, 90%, 95%, 96%, 97%, 98%, and 99%, and most preferably 100%) complementary to contiguous bases of the same length in a target nucleic acid sequence. Complementarity between sequences may be expressed a number of base mismatches in each set of at least 10 contiguous bases being compared.

The terms “polynucleotides”, “nucleic acids”, “nucleotides”, “probes” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. “Polynucleotide” may also be used to refer to peptide nucleic acids (PNA), locked nucleic acids (LNA), threofuranosyl nucleic acids (TNA) and other unnatural nucleic acids or nucleic acid mimics. Other base and backbone modifications known in the art are encompassed in this definition. See, e.g. De Mesmaeker et al (1997) Pure & Appl. Chem., 69, 3, pp 437-440.

The terms “polypeptide”, “peptide” and “protein” are used interchangeably herein to refer to polymers of amino acids of any length. The polymer may be linear, cyclic, or branched, it may comprise modified amino acids, and it may be interrupted by non-amino acids. The terms also encompass amino acid polymers that have been modified, for example, via sulfonation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The term “antibody” as used herein refers to immunoglobulin molecules and immunologically active portions of immunoglobulin molecules, i.e., molecules that contain an antigen-binding site which specifically binds (“immunoreacts with”) an antigen. Structurally, the simplest naturally occurring antibody (e.g., IgG) comprises four polypeptide chains, two heavy (H) chains and two light (L) chains inter-connected by disulfide bonds. The immunoglobulins represent a large family of molecules that include several types of molecules, such as IgD, IgG, IgA, IgM and IgE. The term “immunoglobulin molecule” includes, for example, hybrid antibodies, or altered antibodies, and fragments thereof. It has been shown that the antigen binding function of an antibody can be performed by fragments of a naturally-occurring antibody. These fragments are collectively termed “antigen-binding units”. Antigen binding units can be broadly divided into “single-chain” (“Sc”) and “non-single-chain” (“Nsc”) types based on their molecular structures.

Also encompassed within the terms “antibodies” are immunoglobulin molecules of a variety of species origins including invertebrates and vertebrates. The term “human” as applies to an antibody or an antigen binding unit refers to an immunoglobulin molecule expressed by a human gene or fragment thereof. The term “humanized” as applies to a non-human (e.g. rodent or primate) antibodies are hybrid immunoglobulins, immunoglobulin chains or fragments thereof which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, rabbit or primate having the desired specificity, affinity and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance and minimize immunogenicity when introduced into a human body. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody may also comprise at least a portion of an immunoglobulin constant region (Fc), typically that of a human immunoglobulin.

A “subject” as used herein refers to a biological entity containing expressed genetic materials. The subject is in various embodiments, a vertebrate. In some embodiments, the subject is a mammal. In other embodiments, the subject is a human.

A “control” is an alternative subject or sample used in an experiment for comparison purposes. A control can be “positive” or “negative”. For example, where the purpose of the experiment is to detect a differentially expressed transcript or polypeptide in cell or tissue affected by a disease of concern, it is generally preferable to use a positive control (a subject or a sample from a subject, exhibiting such differential expression and syndromes characteristic of that disease), and a negative control (a subject or a sample from a subject lacking the differential expression and clinical syndrome of that disease.

### Methods

In the first step of one embodiment of the invention, a target nucleic acid present in a sample is contacted with a reaction mixture comprising a nucleic acid probe which, upon binding, exposes a free 3′ end which is capable of being extended by a polymerase. The nucleic acid probe comprises a sequence which is complementary to that of the target sequence, such that the probe specifically anneals to the template. The free 3′ end of the probe subsequently hybridizes to a nucleic acid template and is extended by a polymerase. The dNTP incorporation activity of the polymerase is subsequently detected.

The reaction mixture comprises a nucleic acid probe capable of binding to and complementary to a target nucleic acid. The nucleic acid probe further comprises a 3′ end which is not extended by a polymerase in the absence of target nucleic acid. In one aspect, the nucleic acid probe comprises a first sequence complementary to a template nucleic acid sequence, and further comprises a second sequence at the 3′ end of the nucleic acid sequence, wherein the second sequence is complementary to a third sequence within the nucleic acid sequence, such that the second sequence and the third sequence are annealed in the absence of template. In one embodiment, the nucleic acid probe forms a hairpin structure.

Upon binding of the nucleic acid probe to the template nucleic acid, the free 3′ end of the nucleic acid probe binds to a complementary sequence of a nucleic acid template molecule. The nucleic acid template may be a single-stranded nucleic acid, a double-stranded nucleic acid, or a partially single-stranded nucleic acid. In some embodiments, the nucleic acid template comprises between 15 and 10000 nt, for example between 15 and 6500 nt; between 15 and 2000 nt; between 15 and 1000 nt; between 15 and 400 nt; between 15 and 200 nt; between 15 and 150 nt; between 15 and 100 nt; or between 15 and 75 nt. In one aspect, the nucleic acid template is linear. In another aspect, the nucleic acid template is a circular oligonucleotide. A circular oligonucleotide of any size may be used, but is generally at least 15 nt long. In some embodiments, the circular oligonucleotide comprises between 15 and 10000 nt, for example between 15 and 6500 nt; between 15 and 2000 nt; between 15 and 1000 nt; between 15 and 400 nt; between 15 and 200 nt; between 15 and 150 nt; between 15 and 100 nt; or between 15 and 75 nt. In some embodiments, the base composition of the nucleic acid template is chosen to favor certain bases. In some embodiments, the nucleic acid template comprises less than 25, 20, 15, 10, or 5% T bases. In some embodiments, the nucleic acid template comprises no T bases. In some embodiments, the nucleic acid template comprises less than 5% T bases and less than 65% G/C bases.

Extension of the free 3′ end of the probe bound to the nucleic acid template is performed by a polymerase. The term “polymerase” refers to an enzyme that is capable of adding at least one nucleotide onto the 3′ end of a primer, or to a primer extension product, that is annealed to a template nucleic acid sequence. In certain embodiments, the nucleotide is added to the 3′ end of the primer in a template-directed manner. In certain embodiments, the polymerase is capable of sequentially adding two or more nucleotides onto the 3′ end of the primer. In certain embodiments, the polymerase is active at 37° C. In certain embodiments, the polymerase is active at a temperature other than 37° C. In certain embodiments, the polymerase is active at a temperature greater than 37° C. In certain embodiments, the polymerase is active at both 37° C. and other temperatures. A “DNA polymerase” catalyzes the polymerization of deoxynucleotides.

The term “thermostable polymerase” refers to a polymerase that retains its ability to add at least one nucleotide onto the 3′ end of a primer, or to a primer extension product, that is annealed to a target nucleic acid sequence at a temperature higher than 37° C. In certain embodiments, the thermostable polymerase remains active at a temperature greater than about 37° C. In certain embodiments, the thermostable polymerase remains active at a temperature greater than about 42° C. In certain embodiments, the thermostable polymerase remains active at a temperature greater than about 50° C. In certain embodiments, the thermostable polymerase remains active at a temperature greater than about 60° C. In certain embodiments, the thermostable polymerase remains active at a temperature greater than about 70° C. In certain embodiments, the thermostable polymerase remains active at a temperature greater than about 80° C. In certain embodiments, the thermostable polymerase remains active at a temperature greater than about 90° C.

In certain embodiments, a polymerase is a processive polymerase. In certain embodiments, a processive polymerase remains associated with the template for two or more nucleotide additions. In certain embodiments, a non-processive polymerase disassociates from the template after the addition of each nucleotide. In certain embodiments, a processive DNA polymerase has a characteristic polymerization rate. In certain embodiments, a processive DNA polymerase has a polymerization rate of between 5 to 300 nucleotides per second. In certain embodiments, a processive DNA polymerase has a higher processivity in the presence of accessory factors. For example, and without limitation, the processivity of a processive DNA polymerase may be influenced by the presence or absence of accessory ssDNA binding proteins and helicases. In some embodiments, the processive DNA polymerase comprises a polymerase subunit fused to one or more accessory factors, such as a ssDNA binding protein or a helicase.

In certain embodiments, a DNA polymerase is a strand displacement polymerase. In certain embodiments, a processive DNA polymerase is also a strand displacement polymerase. A strand displacement polymerase is capable of displacing a hybridized strand encountered during replication. In certain embodiments, a strand displacement polymerase requires a strand displacement factor to be capable of displacing a hybridized strand encountered during replication. A “strand displacement factor” is a factor that facilitates strand displacement. In certain embodiments, a strand displacement polymerase is capable of displacing a hybridized strand encountered during replication in the absence of a strand displacement factor. In certain embodiments, the strand displacement polymerase lacks 5′ to 3′ exonuclease activity.

In some embodiments, the DNA polymerase is selected from the group consisting of an A family DNA polymerase; a B family DNA polymerase; a mixed-type polymerase; an unclassified DNA polymerase and RT family polymerase; and variants and derivatives thereof. In some embodiments, the DNA polymerase is an A family DNA polymerase selected from the group consisting of a Pol I-type DNA polymerase such as E. coli DNA polymerase, the Klenow fragment of E. coli DNA polymerase, Bst DNA polymerase, Taq DNA polymerase, Platinum Taq DNA polymerase series, T7 DNA polymerase, and Tth DNA polymerase. In some embodiments, the DNA polymerase is Bst DNA polymerase. In other embodiments, the DNA polymerase is E. coli DNA polymerase. In some embodiments, the DNA polymerase is the Klenow fragment of E. coli DNA polymerase. In some embodiments, the polymerase is Taq DNA polymerase. In some embodiments, the polymerase is T7 DNA polymerase.

In other embodiments, the DNA polymerase is a B family DNA polymerase selected from the group consisting of Bst polymerase, Tli polymerase, Pfu polymerase, Pfu Turbo polymerase, Pyrobest polymerase, Pwo polymerase, KOD polymerase, Sac polymerase, Sso polymerase, Poc polymerase, Pab polymerase, Mth polymerase, Pho polymerase, ES4 polymerase, VENT polymerase, DEEPVENT polymerase, Therminator™ polymerase, phage phi29 polymerase, and phage B103 polymerase. In some embodiments, the polymerase is KOD polymerase. In some embodiments, the polymerase is Therminator™ polymerase. In some embodiments, the polymerase is phage Phi29 DNA polymerase. In some embodiments, the polymerase is Bst, Bst 2.0 or Bst 3.0 polymerase.

In other embodiments, the DNA polymerase is a mixed-type polymerase selected from the group consisting of EX-Taq polymerase, LA-Taq polymerase, Expand polymerase series, and Hi-Fi polymerase. In yet other embodiments, the DNA polymerase is an unclassified DNA polymerase selected from the group consisting of Tbr polymerase, Tfl polymerase, Tru polymerase, Tac polymerase, Tne polymerase, Tma polymerase, Tih polymerase, and Tfi polymerase.

In some embodiments, the DNA polymerase is Q5™ polymerase. In other embodiments, the DNA polymerase is Phusion™ polymerase. In some embodiments, the DNA polymerase is a Bst DNA polymerase.

In other embodiments, the DNA polymerase is an RT polymerase selected from the group consisting of HIV reverse transcriptase, M-MLV reverse transcriptase and AMV reverse transcriptase. In some embodiments, the polymerase is HIV reverse transcriptase or a fragment thereof having DNA polymerase activity.

In some embodiments, a blend of polymerases is used.

In certain embodiments, a reaction composition comprises strand displacement factors. Exemplary strand displacement factors include, but are not limited to, helicases and single stranded DNA binding protein. In certain embodiments, the temperature of the reaction affects strand displacement. In certain embodiments, a temperature of approximately 40° C., 45° C., 50° C., 55° C., 60° C., 65° C., 70° C., 75° C., 80° C., 85° C., or 90° C. facilitates strand displacement by allowing segments of double stranded DNA to separate and reanneal.

In certain embodiments, a reaction composition includes additives. Exemplary additives that may be included in an amplification reaction include, but are not limited to, betaine, formamide, KCl, CaCl2, MgOAc, MgCl2, NaCl, NH4OAc, NaI, Na(CO3)2, LiCl, MnOAc, NMP, Trehalose, DMSO, Glycerol, Ethylene Glycol, Propylene Glycol, Glycinamide, CHES, Percoll, Aurintricarboxylic acid, Tween-20, Tween 21, Tween 40, Tween 60, Tween 85, Brij 30, NP-40, Triton X-100, CHAPS, CHAPSO, Mackernium, LDAO, Zwittergent 3-10, Zwittergent 3-14, Zwittergent SB 3-16, Empigen, NDSB-20, pyrophosphatase, T4 gene 32 protein, E. coli SSB, RecA, nicking endonucleases, 7-deazaG, anionic detergents, cationic detergents, non-ionic detergents, zwittergent, sterol, osmolytes, cations, and any other chemical, protein, or cofactor that may alter the efficiency of nucleic acid extension. In certain embodiments, two or more additives are included in an amplification reaction.

A detection method is used which is sensitive to the nucleotide incorporation activity of the polymerase. The detection step can occur in parallel with the activity of the polymerase, or the detection may be performed subsequent to the polymerase extension step.

In one aspect, the detection occurs by detection of the pyrophosphate (“PPi”) product resulting from dNTP incorporation. Alternatively, a non-natural dNTP is used which, upon incorporation by polymerase, releases a detectable byproduct such as ATP.

PPi detection may, for example, be accomplished by detecting ATP produced from APS in the presence of an enzymatic catalyst. One such catalyst is ATP sulfurylase, which quantitatively converts PPi to ATP in the presence of adenosine 5′ phosphosulfate (APS). Thus, in one embodiment, PPi can be converted to ATP, and the amount of ATP can be measured as discussed below to determine the amount of dNTP incorporated during the reaction.

In another aspect, the detection uses an ATP-releasing nucleotide (“ARN”) which, upon incorporation by polymerase, releases a molecule of ATP. In one embodiment, the ARN has the formula:

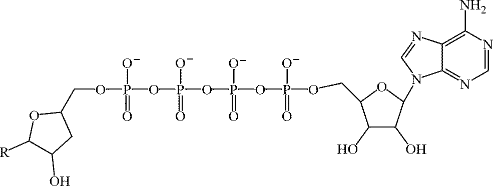

wherein R is any purine or pyrimidine, or or an analog thereof that retains an ability to base pair with a complementary nucleotide. In some embodiments, one of the following ARNs is used:

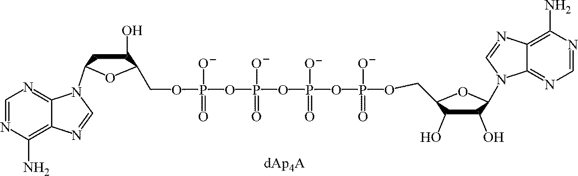

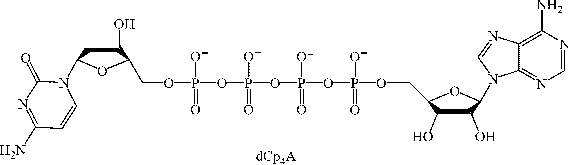

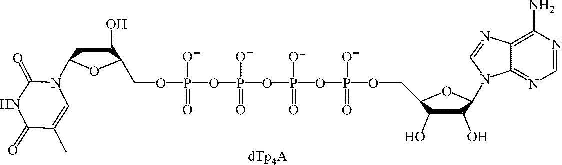

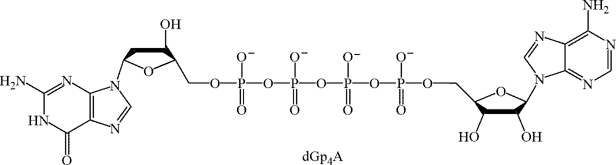

ARNs are described, for example, in US Application No. 2017/0159112; and Mohsen, Michael G., Debin Ji, and Eric T. Kool. “Polymerase-amplified release of ATP (POLARA) for detecting single nucleotide variants in RNA and DNA.” Chemical science 10.11 (2019): 3264-3270.

ATP can be quantified to measure the incorporation of dNTPs. ATP drives the luciferase-mediated conversion of luciferin to oxyluciferin that generates visible light in quantities that are proportional to the quantity of ATP. The light produced in the luciferase-catalyzed reaction may be detected, e.g., by a charge coupled device (CCD) camera, photodiode and/or photomultiplier tube (PMT). Light signals are proportional to the number of nucleotides incorporated. Detected signal can be translated into a system output corresponding to the results which is viewable by a user.

In some aspects, an ATP degrading enzyme, such as apyrase, is used to degrade ATP already present in a sample. For example, a sample is treated with apyrase prior to being contacted with a reagent mixture of the invention. In some embodiments, the apyrase is immobilized on a solid support, such as a container surface or a bead.

In some aspects, a PPi degrading enzyme, such as pyrophosphatase, is used to degrade PPi already present in a sample. For example, a sample is treated with pyrophosphatase prior to being contacted with a reagent mixture of the invention. In some embodiments, the pyrophosphatase is immobilized on a solid support, such as a container surface or a bead.

In some aspects, the reaction composition comprises a dNTP analog, for example a dATP analog. The dATP analog includes any analog that is a poor substrate for luciferase. Such dATP analogs include, but are not limited to dATPαS, 7-deaza-dATP, N⁶-methyl-dATP, 7-deaza-7-propargylamino-dATP, 2-amino-dATP, 2-aminopurine-drTP, and dITP:

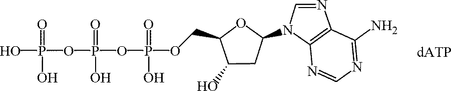 [SMILES: Nc1ncnc2c1ncn2[C@H]1C[C@H](O)[C@@H](COP(=O)(O)OP(=O)(O)OP(=O)(O)O)O1 ; "dATP"]

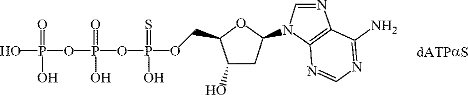 [SMILES: "dATPaS" ; Nc1ncnc2c1ncn2[C@H]1C[C@H](O)[C@@H](COP(O)(=S)OP(=O)(O)OP(=O)(O)O)O1]

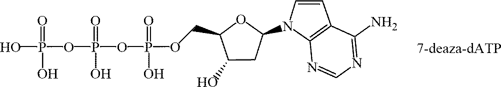 [SMILES: Nc1ncnc2c1ccn2[C@H]1C[C@H](O)[C@@H](COP(=O)(O)OP(=O)(O)OP(=O)(O)O)O1 ; "7-deaza-dATP"]

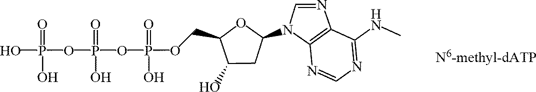 [SMILES: CNc1ncnc2c1ncn2[C@H]1C[C@H](O)[C@@H](COP(=O)(O)OP(=O)(O)OP(=O)(O)O)O1 ; "N6-methyl-dATP"]

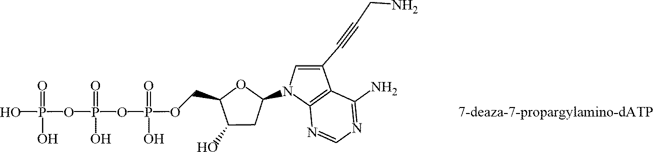 [SMILES: NCC#Cc1cn([C@H]2C[C@H](O)[C@@H](COP(=O)(O)OP(=O)(O)OP(=O)(O)O)O2)c2ncnc(N)c12 ; "7-deaza-7-propargylamino-dATP"]

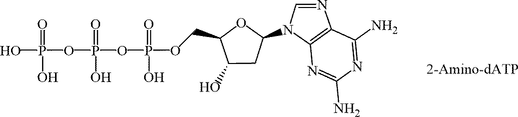 [SMILES: Nc1nc(N)c2ncn([C@H]3C[C@H](O)[C@@H](COP(=O)(O)OP(=O)(O)OP(=O)(O)O)O3)c2n1 ; "2-Amino-dATP"]

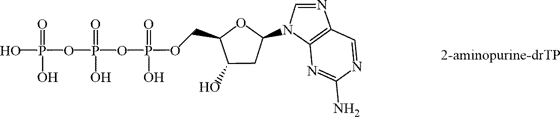 [SMILES: Nc1ncc2ncn([C@H]3C[C@H](O)[C@@H](COP(=O)(O)OP(=O)(O)OP(=O)(O)O)O3)c2n1 ; "2-aminopurine-drTP"]

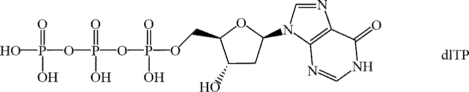 [SMILES: O=c1[nH]cnc2c1ncn2[C@H]1C[C@H](O)[C@@H](COP(=O)(O)OP(=O)(O)OP(=O)(O)O)O1 ; "dlTP"]

### Devices

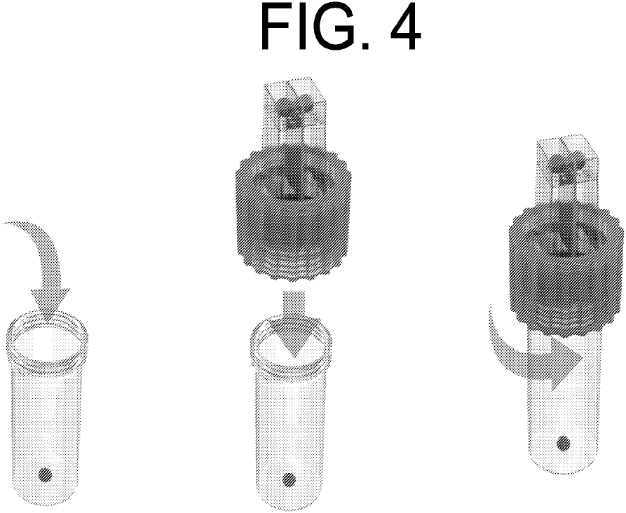

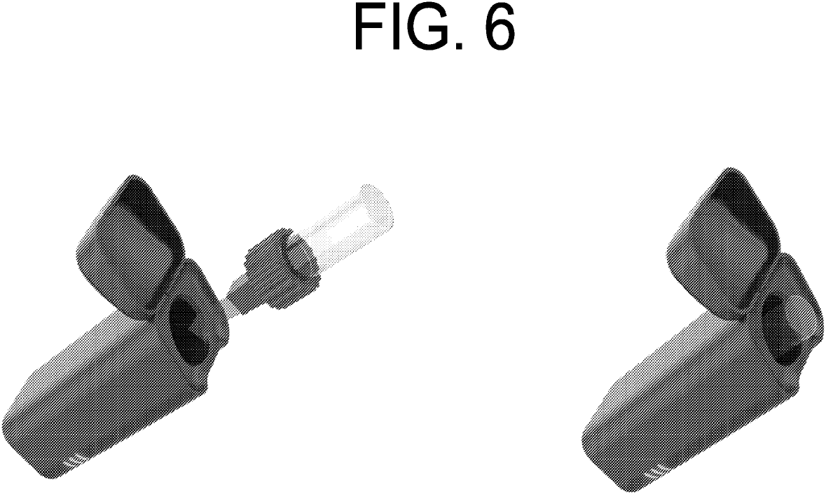

The invention further provides devices for performing the methods of the invention. In one aspect, a device is provided comprising an optical sensor, for example a photomultiplier tube (“PMT”) or a photodiode (e.g. an avalanche photodiode “APD”). The device may further comprise a heater. The device is configured such that it is capable of accepting a consumable sample cartridge comprising the sample to be analyzed and any needed reagents (FIG. 6). In one embodiment, the cartridge comprises a reservoir or vial for collecting a biological sample. For example, the reservoir is configured for storage of saliva or another biological liquid. The reservoir may comprise reagents for pretreatment of the sample, for example immobilized reagents to reduce preexisting PPi or ATP concentrations, or reagents for lysing cells or viral particles present in the samples (FIG. 4). In one embodiment, the reservoir is attached to a cap configured to close the reservoir. The cap may comprise one or more reaction chambers for performing the methods of the invention. In one embodiment, the reaction chamber comprises compositions for performing the methods of the invention. For example, a reagent mixture for nucleic acid amplification is provided comprising a nucleic acid probe, a nucleic acid template, a polymerase, dNTPs and/or a buffer. The reaction mixture may also comprise reagents for detection, for example a reagent mixture comprising luciferase, a luciferase substrate, and/or a buffer. In some embodiments, the same reagent mixture is used for nucleic acid amplification and for detection.

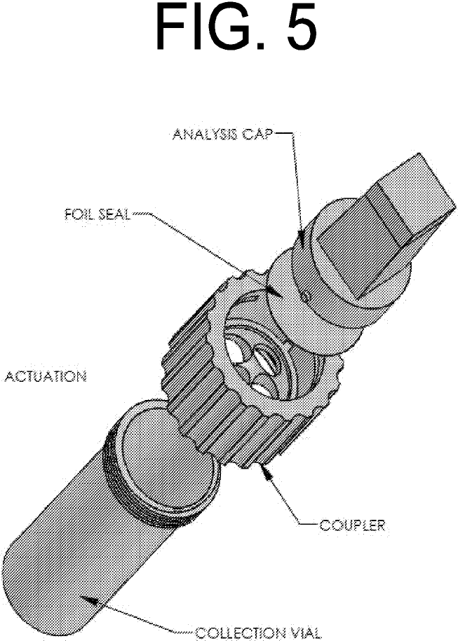

The cap may be connected to the collection reservoir by a mechanical coupler (FIG. 5). The reservoir may be separated from the cap by the presence of a seal, which allows temporary separation of the sample and the reagents in the cap. The analysis can then be started by the action of an actuator located in the cap, which punctures the seal and allows the sample to flow into the reaction chamber(s) and initiate the amplification and detection steps.

The methods of the invention may also be carried out, using, for example, a lateral flow device. Such a lateral flow device may comprise a carrier that allows a lateral flow of the sample from one location on the carrier to another. An example lateral flow carrier may comprise a sample pad, which is an absorbent pad to which the test sample is applied. The carrier may further comprise one or more pretreatment pad(s), which are areas containing immobilized reagents allowing pretreatment of the sample, for example to reduce preexisting PPi or ATP concentrations. The carrier may further contain a reagent pad, comprising the reagents to perform the amplification and detection of the target nucleic acid. The carrier may also comprise a wick or waste reservoir to draw the sample across the carrier by capillary action and to collect it. The lateral flow device also contains an optical detector capable of measuring the signal emitted by the reaction. The lateral flow device may also comprise a heater to control the temperature of the reagent pad.

Devices of the invention may also comprise a microcontrolller, communication ports, and/or a display. In one embodiment, the device communicates wirelessly with a device such as a smartphone.

### Uses

The method disclosed herein can be used for detecting various target nucleic acids of interest. The strand can be a part of a double stranded nucleic acid or a single-stranded nucleic acid. In some embodiments, the target nucleic acid strand can be one present in a cell of a subject, such as a mammal (e.g., human), a plant, a fungus (e.g., a yeast), a protozoa, a bacterium, or a virus. For example, the target nucleic acid can be present in the genome of an organism of interest (e.g., on a chromosome) or on an extrachromosomal nucleic acid. In some embodiments, the target nucleic acid can be RNA, e.g., an mRNA or miRNA. In some other embodiments, the target nucleic acid can be DNA (e.g., double-stranded DNA).

In some embodiments, the target nucleic acid can be a viral nucleic acid. For example, the viral nucleic acid can be a coronavirus (e.g. severe acute respiratory syndrome coronavirus 2 “SARS-CoV-2”), human immunodeficiency virus (HIV), an influenza virus (e.g., an influenza A virus, an influenza B virus, or an influenza C virus), or a dengue virus. Exemplary SARS-CoV-2 target nucleic acids include the ORF1a, ORF1b, S, or N regions. Exemplary HIV target nucleic acids include sequences found in the Pol region.

The target nucleic acid can be present in a bacterium, e.g., a Gram-positive or a Gram-negative bacterium. Examples of the bacterium include a species of a bacterial genus selected from Acinetobacter, Aerococcus, Bacteroides, Bordetella, Campylobacter, Clostridium, Corynebacterium, Chlamydia, Citrobacter, Enterobacter, Enterococcus, Escherichia, Helicobacter, Haemophilus, Klebsiella, Legionella, Listeria, Micrococcus, Mobilincus, Moraxella, Mycobacterium, Mycoplasma, Neisseria, Oligella, Pasteurella, Prevotella, Porphyromonas, Pseudomonas, Propionibacterium, Proteus, Salmonella, Serratia, Staphylococcus, Streptococcus, Treponema, Bacillus, Francisella, or Yersinia.

In some embodiments, the target nucleic acid can be a protozoan nucleic acid. For example, the protozoan nucleic acid can be found in Plasmodium spp., Leishmania spp., Trypanosoma brucei gambiense, Trypanosoma brucei rhodesiense, Trypanosoma cruzi, Entamoeba spp., Toxoplasma spp., Trichomonas vaginalis, and Giardia duodenalis.

In some embodiments, the target nucleic acid is a fungal (e.g., yeast) nucleic acid. For example, the fungal nucleic acid can be found in Candida spp. (e.g., Candida albicans).

In some other embodiments, the target nucleic acid can be a mammalian (e.g., human) nucleic acid. For example, the mammalian nucleic acid can be found in circulating tumor cells, epithelial cells, or fibroblasts. In one example, the target strand is one containing a particular variant, such as single-nucleotide polymorphism (SNP) or a genetic mutation. Examples of such a mutation include a translocation or an inversion.

In some embodiments, the sample to be tested is a bodily fluid, such as blood, plasma, saliva, nasopharyngeal swap (NP swab), nasal swab, oropharyngeal swab, throat swab, bronchoalveolar lavage sample, bronchial aspirate, bronchial washe, endotracheal aspirate, endotracheal wash, tracheal aspirate, nasal secretion sample, mucus sample, or sputum sample. In some embodiments, the biological sample to be tested is saliva. In other embodiments, the biological sample to be tested is a swab, for example a nasal swab, nasopharyngeal swab, buccal swab, oral fluid swab, stool swab, tonsil swab, vaginal swab, cervical swab, blood swab, wound swab, or tube containing blood, sputum, purulent material, or aspirates. In some embodiments, a swab sample is placed in a buffer.

Non-limiting exemplary commercial buffers include the viral transport medium provided with the GeneXpert® Nasal Pharyngeal Collection Kit (Cepheid, Sunnyvale, Calif.); universal transport medium (UTM™, Copan, Murrieta, Calif.); universal viral transport medium (UVT, BD, Franklin Lakes, N.J.); M4, M$RT, M5, and M6 (Thermo Scientific). Further nonlimiting exemplary buffers include liquid Amies medium, PBS/0.5% BSA, PBS/0.5% gelatin, Bartel BiraTrans™ medium, EMEM, PBS, EMEM/1% BSA, sucrose phosphate, Trypticase™ soy broth (with or without 0.5% gelatin or 0.5% BSA), modified Stuart’s medium, veal infusion broth (with or without 0.5% BSA), and saline.

In some embodiments, the sample to be tested is obtained from an individual who has one or more COVID-19 infection symptoms. Nonlimiting exemplary symptoms of influenza include fever, chills, cough, sore throat, runny nose, nasal congestion, muscle ache, headache, fatigue, vomiting, diarrhea, and combinations of any of those symptoms. In some embodiments, the sample to be tested is obtained from an individual who has previously been diagnosed with COVID-19. In some such embodiments, the individual is monitored for recurrence of COVID-19.

In some embodiments, methods described herein can be used for routine screening of healthy individuals with no risk factors. In some embodiments, methods described herein are used to screen asymptomatic individuals, for example, during routine or preventative care. In some embodiments, methods described herein are used to screen women who are pregnant or who are attempting to become pregnant.

### EXAMPLES

### Example 1 Synthesis of a Circular Oligonucleotide Template

Circular oligonucleotides were synthesized according to the general methods known in the art. See, e.g. Diegelman, Amy M., and Eric T. Kool. “Chemical and Enzymatic Methods for Preparing Circular Single-Stranded DNAs.” Current Protocols in Nucleic Acid Chemistry 1 (2000): 5-2. Oligonucleotides were synthesized by Integrated DNA Technologies, Inc.

Briefly, a ligation mixture was prepared comprising (all concentrations final): S54_pre 5′-phosphorylated precursor (CACTCCACTCACAACATCCACACCTCACACTACAACTCCAACACACTCACTCCT, 15 nmol), a splint oligonucleotide (GGAGTGAGGAGT, 45 nmol), MgCl₂ (5 mM), Tris (50 mM), ATP (50 µM), DTT (10 mM), T4 DNA Ligase (0.5 U/µL), and water to 10 mL. The precursor, splint and MgCl₂ were first heated to 90° C. for 20 mins. in a heatblock wrapped in insulating material, then cooled slowly to room temperature. The DTT, ligase and ATP were then added. The ligation was performed at room temperature for 16 h. Upon completion, the reaction mixture was dialyzed in MWCO 3500 SnakeSkin tubing (ThermoFisher) in 3 L of water with 3x water changes (6 hours each). The dialyzed reaction mixture was evaporated to dryness, resuspended in Tris-Urea loading buffer, and purified by polyacrylamide gel electrophoresis (10%). The bands corresponding to ligated material were excised and purified by electroelution, followed by a second dialysis step (3 L water, 3x water changes, 6 h each). After drying and resuspending in water, the final sample was quantitated by Nanodrop One and estimated to have a concentration of 45.6 ng/µL.

### Example 2 Rolling Circle Elongation of a Nucleic Acid Primer Using ARNs

A 20 µL reaction mixture was prepared (all concentrations final) comprising Thermopol buffer (1X), SCR54 circular oligonucleotides (10 nM), primer (GAGTTGTAGTGTGAGG, 20 nM), dGTP (400 µM), dTp4A (ARN, 2 µM), dAp4A (ARN, 2 µM) and an appropriate amount of polymerase (Bst large fragment, 1000U/reaction, or 2 U/reaction of Cell Data Sciences E1, E2, E3 thermostable polymerases). Reactions were incubated at 65° C. for 5 minutes, then cooled to 4° C.

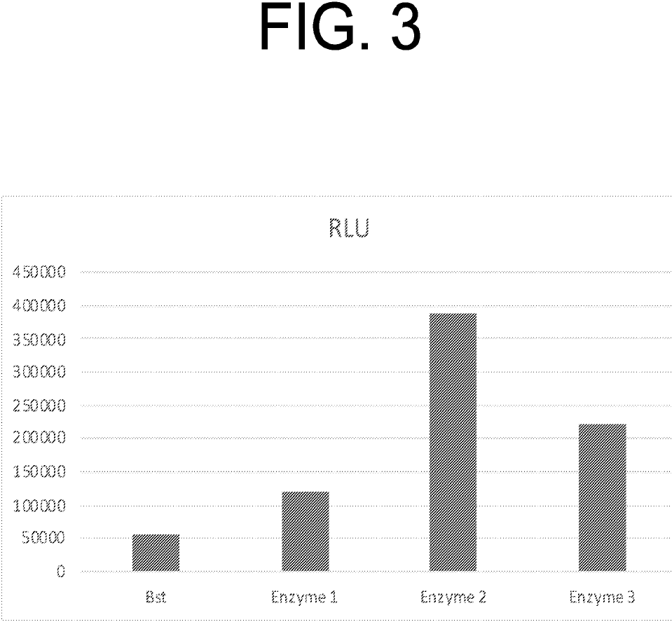

A Promega Enliten ATP Assay Kit was used for ATP detection using a Berthold Lumat LB9507 luminometer. The results of this assay are shown in FIG. 3.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of detecting a target nucleic acid sequence, comprising:
a. contacting a sample suspected to contain the target nucleic acid sequence with a reaction mixture comprising:
i) a first nucleic acid probe comprising a first sequence complementary to a template nucleic acid sequence, and further comprising a sequence P at the 3′ end of the first nucleic acid probe, wherein the sequence P is complementary to a sequence Pc within the first nucleic acid probe, and P is annealed to Pc in the absence of target nucleic acid;
ii) the template nucleic acid comprising the sequence Pc, such that the sequence P anneals to the sequence Pc in the template nucleic acid upon said contacting the template nucleic acid;
iii) a polymerase capable of extending the 3′ end of the nucleic acid probe; and
iv) a plurality of nucleotides capable of being incorporated by the polymerase, thereby extending the 3′ end of the first nucleic acid probe.
b. detecting the activity of the polymerase.

2. The method of claim 1, wherein at least one of the plurality of the nucleotides is an ATP-linked nucleotide, such that incorporation of the ATP-linked nucleotide by the polymerase results in release of a molecule of ATP.

3. The method of claim 2, wherein the ATP-linked nucleotide has the formula: 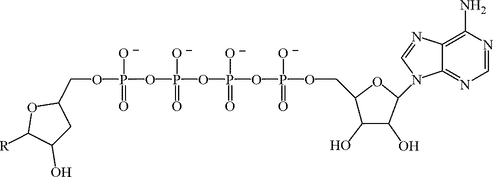
wherein R is a purine, a pyrimidine, or a non-natural base analog.

4. The method of claim 3, wherein R is adenine, guanidine, cytidine or thymidine.

5. The method of claim 2, wherein the detecting comprises measuring the amount of ATP generated by the incorporation of the nucleotide by the polymerase.

6. The method of claim 1, wherein the detecting comprises measuring the amount of pyrophosphate generated by the polymerase.

7. The method of claim 6, wherein the reagent mixture comprises ATP sulfurylase.

8. The method of claim 7, wherein the reagent mixture comprises adenosine 5′-phosphosulfate.

9. The method of claim 6, wherein the reagent mixture comprises a dNTP analog.

10. The method of claim 9, wherein the dNTP analog is a dATP analog.

11. The method of claim 10, wherein the dATP analog is to dATPαS, 7-deaza-dATP, N⁶-methyl-dATP, 7-deaza-7-propargylamino-dATP, 2-amino-dATP, 2-aminopurine-drTP, or dITP.

12. The method of claim 11, wherein the dATP analog is dATPαS.

13. The method of claim 1, wherein the detecting comprises measuring a luminescent signal.

14. The method of claim 13, wherein the reagent mixture comprises luciferase and a luciferase substrate.

15. The method of claim 1, wherein the detecting comprises measuring a fluorescent signal.

16. The method of claim 15, wherein the fluorescent signal results from the presence of a nucleic acid binding dye.

17. The method of claim 6, wherein the detecting comprises electrochemical detection of pyrophosphate.

18. The method of claim 1, wherein the nucleic acid template is DNA.

19. The method of claim 1, wherein the nucleic acid template is linear.

20. The method of claim 1, wherein the nucleic acid template is circular.

21. The method of claim 20, wherein the nucleic acid template is a circular oligonucleotide.

22. The method of claim 1, wherein the circular oligonucleotide comprises between 15 and 200 nt.

23. The method of claim 1, wherein the circular oligonucleotide comprises between 15 and 150 nt.

24. The method of claim 1, wherein the circular oligonucleotide comprises between 15 and 100 nt.

25. The method of claim 1, wherein the circular oligonucleotide comprises between 15 and 75 nt.

26. The method of claim 1, wherein the circular oligonucleotide comprises less than 25, 20, 15, 10, or 5% T bases.

27. The method of claim 26, wherein the circular oligonucleotide comprises no T bases.

28. The method of claim 1, wherein the first nucleic acid probe forms a hairpin.

29. The method of claim 1, wherein the contacting is performed at room temperature.

30. The method of claim 1, wherein the contacting is performed at a temperature greater than 37° C.

31. The method of claim 30, wherein the temperature is between 42 and 70° C.

32. The method of claim 30, wherein the temperature is between 50 and 65° C.

33. The method of claim 1, wherein the polymerase is a thermostable polymerase.

34. The method of claim 1, wherein the reaction mixture further comprises a second nucleic acid probe, wherein the second nucleic acid probe binds to a sequence complementary to that of the circular nucleic acid.

35. The method of claim 1, wherein the reaction mixture further comprises a single-stranded binding protein, for example T4 gene 32 protein.

36. The method of claim 1, wherein prior to the contacting step, the sample is incubated with a reagent that reduces the concentration of ATP.

37. The method of claim 36, wherein the reagent is apyrase.

38. The method of claim 36, wherein the apyrase is immobilized on a solid support.

39. The method of claim 1, wherein prior to the contacting step, the sample is incubated with a reagent that reduces the concentration of pyrophosphate.

40. The method of claim 39, wherein the reagent is pyrophosphatase.

41. The method of claim 39, wherein the pyrophosphatase is immobilized on a solid support.

42. The method of claim 1, wherein the target nucleic acid is RNA, for example SARS-CoV-2 RNA.

43. The method of claim 1, wherein the reaction mixture further comprises a hyperbranching primer.

44. A device configured for performing the method of claim 1.


SEQUENCE LISTING (3 sequences)

SEQ ID NO: 1 - DNA, 54 bp, synthetic construct
  misc_feature 1..54: note = Description of Artificial Sequence: Synthetic oligonucleotide
  source 1..54: mol_type = other DNA
cactccactcacaacatccacacctcacactacaactccaacacactcactcct

SEQ ID NO: 2 - DNA, 12 bp, synthetic construct
  misc_feature 1..12: note = Description of Artificial Sequence: Synthetic oligonucleotide
  source 1..12: mol_type = other DNA
ggagtgaggagt

SEQ ID NO: 3 - DNA, 16 bp, synthetic construct
  misc_feature 1..16: note = Description of Artificial Sequence: Synthetic primer
  source 1..16: mol_type = other DNA
gagttgtagtgtgagg